(19) United States
(12) Patent Application Publication
(10) Pub. No.: US 2014/0296280 A1
(43) Pub. Date: Oct. 2, 2014

(54) PURES-(-)-9-FLUORO-6,7-DIHYDRO-8-(4-HYDROXYPIPERIDIN-1-YL)-5-METHYL-1-OXO-1H,-5H-BENZO[I,J]QUINOLIZINE-2- CARBOXYLIC ACID L-ARGININE SALT TETRAHYDRATE AND A PROCESS FOR ITS PREPARATION

(71) Applicants: Kiran Kumar Gangakhedkar, Aurangabad (IN); Aniruddha Varangaonkar, Aurangabad (IN); Furqan Mohammed Diwan, Aurangabad (IN); Ravindra Dattatraya Yeole, Aurangabad (IN); Keshav Deo, Aurangabad (IN)

(72) Inventors: Kiran Kumar Gangakhedkar, Aurangabad (IN); Aniruddha Varangaonkar, Aurangabad (IN); Furqan Mohammed Diwan, Aurangabad (IN); Ravindra Dattatraya Yeole, Aurangabad (IN); Keshav Deo, Aurangabad (IN)

(21) Appl. No.: 14/240,400

(22) PCT Filed: Jan. 6, 2012
(86) PCT No.: PCT/IB2012/050074

§ 371 Date: May 14, 2014

(30) Foreign Application Priority Data
Sep. 28, 2011 (IN) 270/MUM/2011

(51) Int. Cl. C07D 455/04; A61K 31/4375

(52) U.S. Cl. CPC C07D 455/04; A61K 31/4375
USPC 514/294; 546/94

(57) ABSTRACT

Pure S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate and a process for its preparation is disclosed.

### FIELD OF THE INVENTION

The invention relates to substantially pure S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate and a process for its preparation.

### BACKGROUND OF THE INVENTION

The compound of Formula (I), chemically known as S-(−)-9-fluoro-8-(4-hydroxy piperidin-1-yl)-5-methyl-6,7-dihydro-1-oxo-1H,5H-benzo[i,j]quinolizine-2-carboxylic acid, belongs to a class of antibacterial agents generally known as fluoroquinolones and is useful in treating a broad range of bacterial infections.

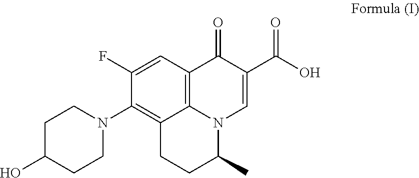 [SMILES: C[C@H]1CCc2c(N3CCC(O)CC3)c(F)cc3c(=O)c(C(=O)O)cn1c23]

U.S. Pat. No. 7,164,023 discloses a crystalline S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate and its preparation.

The present invention is directed to a substantially pure S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate and a process for its preparation.

### SUMMARY OF THE INVENTION

Accordingly, there is provided a substantially pure S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate and a process for it's preparation.

In one general aspect, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w as determined by HPLC.

In another general aspect, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 9-fluoro-8-hydroxy-5-methyl-6,7-dihydro-1-oxo-1H,5H-benzo[i,j]quinolizine-2-carboxylic acid, as determined by HPLC.

In yet another general aspect, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 8-fluoro-9-(4-hydroxy-piperidin-1-yl)-5-methyl-6,7-dihydro-1-oxo-1H,5H-benzo[i,j]quinolizine-2-carboxylic acid, as determined by HPLC.

In another general aspect, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 8-(4-hydroxy-1-piperidinyl)-5-methyl-6,7-dihydro-1-oxo-1H,5H-benzo[i,j]quinolizine-2-carboxylic acid, as determined by HPLC.

In another general aspect, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 8,9-difluoro-5-methyl-6,7-dihydro-1-oxo-1H,5H-benzo[i,j]quinolizine-2-carboxylic acid, determined by HPLC.

In another general aspect, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.5% w/w of R-(+)-9-fluoro-8-(4-hydroxy-piperidin-1-yl)-5-methyl-6,7-dihydro-1-oxo-1H,5H-benzo[i,j]quinolizine-2-carboxylic acid, as determined by HPLC.

In another general aspect there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing bacterial endotoxins in an amount less than 0.35 USP Endotoxin Unit per mg.

In yet another general aspect there is provided a pharmaceutical composition comprising S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate according to the invention.

In another general aspect, there is provided a process for preparing S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w as determined by HPLC, said process comprising:

(a) dissolving L-arginine in a mixture of acetone and water;

(b) adding S-(−)-9-fluoro-6,7-dihydro-8-(4-hydroxypiperidin-1-yl)-5-methyl-1-oxo-1H,-5H-benzo[i,j]quinolizine-2-carboxylic acid to the solution obtained in step (a);

(c) warming the solution obtained in step (b) to get a clear solution;

(d) optionally treating the warm solution obtained in step (c) with activated carbon and filtering the treated solution;

(e) diluting the warm solution obtained in step (d) with acetone and refluxing the diluted solution;

(f) cooling the solution obtained in step (e) to obtain solid S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate; and

(g) collecting S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate obtained in step (f) by filtration, and optionally washing it with acetone.

The details of one or more embodiments of the inventions are set forth in the description below. Other features, objects and advantages of the inventions will be apparent from the following description including claims

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention. It must be noted that, as used in this specification and the appended claims, the singular forms “a,” “an,” and “the” include plural referents unless the content clearly dictates otherwise. All references including patents, patent applications, and literature cited in the specification are expressly incorporated herein by reference in their entirety.

The invention provides substantially pure S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate and a process for its preparation.

The term “substantially pure S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate” as used herein refers to S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having one or more of the following specifications:

(i) S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w as determined by HPLC;

(ii) S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 9-fluoro-8-hydroxy-5-methyl-6,7-dihydro-1-oxo-1H,5H-benzo[i,j]quinolizine-2-carboxylic acid, as determined by HPLC;

(iii) S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 8-fluoro-9-(4-hydroxypiperidin-1-yl)-5-methyl-6,7-dihydro-1-oxo-1H,5H-benzo[i,j]quinolizine-2-carboxylic acid, as determined by HPLC;

(iv) S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 8-(4-hydroxy-1-piperidinyl)-5-methyl-6,7-dihydro-1-oxo-1H,5H-benzo[i,j]quinolizine-2-carboxylic acid, as determined by HPLC;

(v) S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 8,9-difluoro-5-methyl-6,7-dihydro-1-oxo-1H,5H-benzo[i,j]quinolizine-2-carboxylic acid, determined by HPLC;

(vi) S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.5% w/w of R-(+)-9-fluoro-8-(4-hydroxypiperidin-1-yl)-5-methyl-6,7-dihydro-1-oxo-1H,5H-benzo[i,j]quinolizine-2-carboxylic acid, as determined by HPLC; or

(vii) S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing bacterial endotoxins in an amount less than 0.35 USP Endotoxin Unit per mg.

In some embodiments, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w as determined by HPLC.

In some other embodiments, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 9-fluoro-8-hydroxy-5-methyl-6,7-dihydro-1-oxo-1H,5H-benzo[i,j]quinolizine-2-carboxylic acid, as determined by HPLC.

In some other embodiments, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 8-fluoro-9-(4-hydroxy-piperidin-1-yl)-5-methyl-6,7-dihydro-1-oxo-1H,5H-benzo[i,j]quinolizine-2-carboxylic acid, as determined by HPLC.

In some other embodiments, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 8-(4-hydroxy-1-piperidinyl)-5-methyl-6,7-dihydro-1-oxo-1H,5H-benzo[i,j]quinolizine-2-carboxylic acid, as determined by HPLC.

In some embodiments, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.15% w/w of 8,9-difluoro-5-methyl-6,7-dihydro-1-oxo-1H,5H-benzo[i,j]quinolizine-2-carboxylic acid, determined by HPLC.

In some embodiments, there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing less than 0.5% w/w of R-(+)-9-fluoro-8-(4-hydroxy-piperidin-1-yl)-5-methyl-6,7-dihydro-1-oxo-1H,5H-benzo[i,j]quinolizine-2-carboxylic acid, as determined by HPLC.

In another general aspect, there is provided a process for preparing S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w as determined by HPLC, said process comprising:

(a) dissolving L-arginine in a mixture of acetone and water;

(b) adding S-(−)-9-fluoro-6,7-dihydro-8-(4-hydroxypiperidin-1-yl)-5-methyl-1-oxo-1H,-5H-benzo[i,j]quinolizine-2-carboxylic acid to the solution obtained in step (a);

(c) warming the solution obtained in step (b) to get a clear solution;

(d) optionally treating the warm solution obtained in step (c) with activated carbon and filtering the treated solution;

(e) diluting the warm solution obtained in step (d) with acetone and refluxing the diluted solution;

(f) cooling the solution obtained in step (e) to obtain solid S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate; and

(g) collecting S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate obtained in step (f) by filtration, and optionally washing it with acetone.

In some embodiments there is provided S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w, further containing bacterial endotoxins in an amount less than 0.35 USP Endotoxin Unit per mg.

In some embodiments, there is provided a pharmaceutical composition comprising substantially pure S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate according to the invention.

In some other embodiments, the pharmaceutical compositions according to the invention may further comprise one or more pharmaceutically acceptable excipients.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Synthesis of Pure S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate

In a typical experiment, 0.45 Kg of L-arginine was dissolved in 7.5 Ltr of mixture of acetone and water (4:3.5). 1 Kg of S-(−)-9-fluoro-6,7-dihydro-8-(4-hydroxypiperidin-1-yl)-5-methyl-1-oxo-1H,-5H-benzo[i,j]quinolizine-2-carboxylic acid was charged to this solution and warmed to 55° C. to obtain a clear solution. The clear solution was treated with activated carbon at 50° C. for 0.5 hours and hot filtered through a 5μ filter and finally through a 0.2μ filter. The filtrate thus obtained was diluted slowly with 9.8 Ltr of acetone (0.2μ filtered) at 50-55° C. The reaction mass thus obtained was refluxed for 0.5 hours and slowly cooled to about 10-15° C. The solid product thus obtained was collected by filtration and washed with acetone. The wet material was vacuum dried at 30-40° C. to get about 1.4 Kg of S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate which was analyzed for content of various components using HPLC and the results are described in Table 1.

The amount of R-(+)-9-fluoro-8-(4-hydroxy-piperidin-1-yl)-5-methyl-6,7-dihydro-1-oxo-1H,5H-benzo[i,j]quinolizine-2-carboxylic acid (or R-(+)-isomer content) was determined using HPLC (Waters 2695 separation module or equivalent). The HPLC column having 250 mm length and 4.6 mm ID packed with 10μ particles of (S,S) Whelk-O1 10/100 was used. Mobile phase used was a mixture of ammonium acetate solution (1.52 gm in 1000 ml of HPLC grade water) and ethanol in ratio of 45:55, v/v. Flow rate of mobile phase was maintained at 1.2 ml/min. Column temperature was maintained at 40° C. Detection was carried out using UV detector at wavelength 295 nm. Standard solution and test solution were prepared in methanol.

The content of other substances (Table 1, Sr. No. 1 to 5) in the product was determined using HPLC (Waters 2695 separation module or equivalent). The HPLC column having 150 mm length and 4.6 mm ID packed with 3.5μ particles of X-Terra RP18 was used. Mobile phase A used was a mixture of ammonium acetate (3.0 gm) and sodium perchlorate monohydrate (8.4 gm) in 1000 ml of HPLC grade water with final pH adjusted to 2.2 with orthophosphoric acid. Mobile phase B was mixture of methanol and acetonitrile in ratio of 60:40, v/v. Mobile phase was run in gradient mode. Initially mobile phase A and B was run at 75:25 for 15 minutes, slowly ratio of mobile phase B was raised to 50% in 20 min, held for 10 minutes at this concentration and brought back to initial condition in next 3 minutes and held for 7 minutes before next run. Flow rate of mobile phase was maintained at 1.2 ml/min. Column temperature was maintained at 40° C. Detection was carried out using UV detector at wavelength 237 nm. Standard solution and test solution were prepared in mixture of water and mobile phase B in ratio of 50:50, v/v with pH adjusted to 8.5 with dilute ammonia.

TABLE 1
Results of HPLC analysis of S-(−)-9-fluoro-6,7-dihydro-8-(4-hydroxypiperidin-
1-yl)-5-methyl-1-oxo-1H, -5H-benzo[i,j]quinolizine-2-carboxylic
acid L-arginine salt tetrahydrate according to the invention

| | | Content (as |
|---|---|---|
| | | determined by |
| Sr. | Component | HPLC) |
| 1. | S-(−)-9-fluoro-6,7-dihydro-8-(4-hydroxypiperidin-1-yl)-5-methyl- | At least 99% |
| | 1-oxo-1H, -5H-benzo[i,j]quinolizine-2-carboxylic acid L-arginine | w/w |
| | salt tetrahydrate. | |
| 2. | 9-fluoro-8-hydroxy-5-methyl-6,7-dihydro-1-oxo-1H,5H-benzo | Less than 0.15% |
| | [i,j]quinolizine-2-carboxylic acid | w/w |
| 3. | 8-fluoro-9-(4-hydroxy-piperidin-1-yl)-5-methyl-6,7-dihydro-1- | Less than 0.15% |
| | oxo-1H,5H-benzo[i,j]quinolizine-2-carboxylic acid | w/w |
| 4. | 8-(4-hydroxy-1-piperidinyl)-5-methyl-6,7-dihydro-1-oxo-1H,5H- | Less than 0.15% |
| | benzo[i,j]quinolizine-2-carboxylic acid | w/w |
| 5. | 8,9-difluoro-5-methyl-6,7-dihydro-1-oxo-1H,5H-benzo[i,j] | Less than 0.15% |
| | quinolizine-2-carboxylic acid | w/w |
| 6. | R-(+)-9-fluoro-8-(4-hydroxy-piperidin-1-yl)-5-methyl-6,7- | Less than 0.5% |
| | dihydro-1-oxo-1H,5H-benzo[i,j]quinolizine-2-carboxylic acid | w/w |
| 7. | Bacterial endotoxins | Less than 0.35 |
| | | USP Endotoxin |
| | | Units per mg |

We claim:

1. S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w as determined by HPLC.

2. S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate according to claim 1, further containing less than 0.15% w/w of 9-fluoro-8-hydroxy-5-methyl-6,7-dihydro-1-oxo-1H,5H-benzo[i,j]quinolizine-2-carboxylic acid as determined by HPLC.

3. S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate according to claim 1, further containing less than 0.15% w/w of 8-fluoro-9-(4-hydroxy-piperidin-1-yl)-5-methyl-6,7-dihydro-1-oxo-1H,5H-benzo[i,j]quinolizine-2-carboxylic acid as determined by HPLC.

4. S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate according to claim 1, further containing less than 0.15% w/w of 8-(4-hydroxy-1-piperidinyl)-5-methyl-6,7-dihydro-1-oxo-1H,5H-benzo[i,j]quinolizine-2-carboxylic acid as determined by HPLC.

5. S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate according to claim 1, further containing less than 0.15% w/w of 8,9-difluoro-5-methyl-6,7-dihydro-1-oxo-1H,5H-benzo[i,j]quinolizine-2-carboxylic acid as determined by HPLC.

6. S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate according to claim 1, further containing less than 0.5% w/w of R-(+)-9-fluoro-8-(4-hydroxypiperidin-1-yl)-5-methyl-6,7-dihydro-1-oxo-1H,5H-benzo[i,j]quinolizine-2-carboxylic acid as determined by HPLC.

7. S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate according to claim 1, further containing bacterial endotoxins in an amount less than 0.35 USP Endotoxin Units per mg.

8. A pharmaceutical composition comprising S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate according to any of the claims 1 to 7.

9. A pharmaceutical composition according to claim 8, further comprising one or more pharmaceutically acceptable excipients.

10. A process for preparing S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate having a purity of at least 99% w/w as determined by HPLC, said process comprising:
(a) dissolving L-arginine in a mixture of acetone and water;
(b) adding S-(−)-9-fluoro-6,7-dihydro-8-(4-hydroxypiperidin-1-yl)-5-methyl-1-oxo-1H,-5H-benzo[i,j]quinolizine-2-carboxylic acid to the solution obtained in step (a);
(c) warming the solution obtained in step (b) to get a clear solution;
(d) optionally treating the warm solution obtained in step (c) with activated carbon and filtering the treated solution;
(e) diluting the warm solution obtained in step (d) with acetone and refluxing the diluted solution;
(f) cooling the solution obtained in step (e) to obtain solid S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate;
(g) collecting S-(−)-9-fluoro-6,7-dihydro-8-(4-hydroxypiperidin-1-yl)-5-methyl-1-oxo-1H,-5H-benzo[i,j]quinolizine-2-carboxylic acid L-arginine salt tetrahydrate obtained in step (f) by filtration, and optionally washing it with acetone.
